(19) United States
(12) Patent Application Publication
(10) Pub. No.: US 2022/0354987 A1
(43) Pub. Date: Nov. 10, 2022

(54) COLLAGEN POLYSACCHARIDE WOUND DRESSING

(71) Applicant: Systagenix Wound Management, Limited, Bracknell (GB)

(72) Inventor: Paul William WATT, West Sussex (GB)

(73) Assignee: Systagenix Wound Management, Limited, Bracknell (GB)

(21) Appl. No.: 17/623,528

(22) PCT Filed: Jun. 25, 2020
(86) PCT No.: PCT/IB2020/056045

§ 371 Date: Dec. 28, 2021

Related U.S. Application Data
(60) Provisional application No. 62/868,510, filed on Jun. 28, 2019.

(51) Int. Cl. A61L 15/44; A61L 15/32; A61L 15/28

(52) U.S. Cl. CPC A61L 15/44; A61L 15/325; A61L 15/28; A61L 2300/104; A61L 2300/414

(57) ABSTRACT

The present disclosure relates generally to wound dressing compositions that include a β-glucan which can stimulate an immunomodulatory effect on cells within a wound, increase the healing rate of a wound, and stimulate the healing of recalcitrant wounds. The wound dressing includes a mixture of a collagen, a polysaccharide, and a β-glucan. Also disclosed herein are methods for use of the wound dressing as well as kits including the wound dressings of the present technology.

### TECHNICAL FIELD

The present technology relates generally to wound dressings that include a mixture of a collagen, a polysaccharide, and a β-glucan. Such wound dressing compositions may be used to expedite healing in a wound upon application.

### BACKGROUND

The following description of the background of the present technology is provided simply as an aid in understanding the present technology and is not admitted to describe or constitute prior art to the present technology.

A wide variety of materials and devices, generally characterized as “dressings,” are generally known in the art for use in treating an injury or other disruption of tissue. Such wounds may be the result of trauma, surgery, or disease, and may affect skin or other tissues. In general, dressings may control bleeding, absorb wound exudate, ease pain, assist in debriding the wound, protect wound tissue from infection, or otherwise promote healing and protect the wound from further damage. Proteases play pivotal roles in normal wound healing processes. In general, different wound-related proteases act on various proteins, including proteins of the extracellular matrix (ECM) and connective tissue. In the normal wound healing process, proteases break down damaged ECM proteins and foreign material so that new tissue can form and wound closure can occur. Excessive wound proteases lead to the breakdown of newly formed ECM and other beneficial proteins such as cytokines and growth factors. As a result, wound healing is impaired due to damage to the ECM, reduction of beneficial proteins, and abnormal prolongation of the inflammatory stage.

### SUMMARY

In an aspect, a wound dressing is provided that includes about 30 wt. % to about 95 wt. % of a collagen, about 30 wt. % to about 70 wt. % of a polysaccharide, and about 0.05 wt. % to about 10 wt. % of a β-glucan.

In a related aspect, a method for treating a subject in need thereof is provided, where the method includes administering to the wound a wound dressing of any embodiment disclosed herein.

In a further related aspect, a kit is provided that includes a wound dressing of any embodiment disclosed herein, as well as instructions for use.

In a further related aspect, a kit is provided that includes a dressing and instructions for making a wound composition of any embodiment disclosed herein, where the dressing includes about 30 wt. % to about 95 wt. % of a collagen and about 30 wt. % to about 70 wt. % of a polysaccharide.

### DETAILED DESCRIPTION

It is to be appreciated that certain aspects, modes, embodiments, variations and features of the present methods are described below in various levels of detail in order to provide a substantial understanding of the present technology.

### Definitions

The definitions of certain terms as used in this specification are provided below. Unless defined otherwise, all technical and scientific terms used herein generally have the same meaning as commonly understood by one of ordinary skill in the art to which this present technology belongs.

The following terms are used throughout as defined below.

As used herein and in the appended claims, singular articles such as “a”, “an”, and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as discloses “10 wt. %.”

As used herein, the “administration” of a wound dressing composition to a subject includes any route of introducing or delivering to a subject a diagnostic wound dressing composition to perform its intended function. Administration can be carried out by any suitable route, including but not limited to, topical administration. Administration includes self-administration and the administration by another.

As used herein, the term “effective amount” refers to a quantity sufficient to achieve a desired therapeutic and/or prophylactic effect, e.g., an amount which results in the decrease in a wound described herein or one or more signs or symptoms associated with a wound described herein. In the context of therapeutic or prophylactic applications, the amount of a composition administered to the subject will vary depending on the composition, the degree, type, and severity of the wound and on the characteristics of the individual. The compositions can also be administered in combination with one or more additional therapeutic compounds. In the methods described herein, the therapeutic compositions may be administered to a subject having one or more wounds.

The term “mammalian recombinant collagen” refers to collagen manufactured by culturing a non-human organism or mammalian or non-mammalian cells to express at least one exogenous gene encoding a collagen in the culturing system. The term “human recombinant collagen” refers to collagen manufactured by culturing a non-human organism or mammalian or non-mammalian cells to express at least one human gene encoding a collagen. The human recombinant collagen may be selected from the group consisting of collagen type I, type II, type III, type IV, type V, type VI, type VII, type VIII, type IX, type X, type XI, type XII, type XIII, type XIV, type XV, type XVI, type XVII, type XVIII, type XIX, type XX, type XXI, type XXII, type XXIII, type XXIV, type XXV, type XXVI, and type XXVII. The human recombinant collagen can be collagen of one type free of any other type, or can be a mixture of collagen types. Suitably, the human recombinant collagen comprises collagens selected from the group consisting of collagen type I, collagen type III, and mixtures thereof. The term “bovine recombinant collagen” refers to collagen manufactured by culturing a non-human organism or mammalian or non-mammalian cells to express at least one bovine gene encoding a collagen. The bovine recombinant collagen may be selected from the group consisting of collagen type I, type II, type III, and type IV. The bovine recombinant collagen can be collagen of one type free of any other type, or can be a mixture of collagen types. Suitably, the bovine recombinant collagen comprises collagens selected from the group consisting of collagen type I, collagen type III, and mixtures thereof.

As understood by one of ordinary skill in the art, “molecular weight” (also known as “relative molar mass”) is a dimensionless quantity but is converted to molar mass by multiplying by 1 gram/mole—for example, collagen with a weight-average molecular weight of 5,000 has a weight-average molar mass of 5,000 g/mol.

As used herein, the terms “individual”, “patient”, or “subject” can be an individual organism, a vertebrate, a mammal, or a human. In some embodiments, the individual, patient or subject is a human.

“Treating” or “treatment” as used herein covers the treatment of a wound described herein, in a subject, such as a human, and includes: (i) inhibiting a wound, i.e., arresting its development; (ii) relieving a wound, i.e., causing regression of the wound; (iii) slowing progression of the wound; and/or (iv) inhibiting, relieving, or slowing progression of one or more symptoms of the wound. In some embodiments, treatment means that the symptoms associated with the wound are, e.g., alleviated, reduced, cured, or placed in a state of remission.

It is also to be appreciated that the various modes of treatment of wounds as described herein are intended to mean “substantial,” which includes total but also less than total treatment, and wherein some biologically or medically relevant result is achieved. The treatment may be a continuous prolonged treatment for a chronic wound or a single, or few time administrations for the treatment of an acute wound.

### The Wound Dressing of the Present Technology

Proteases play pivotal roles in normal wound healing processes. In general, different wound-related proteases act on various proteins, including proteins of the extracellular matrix (ECM) and connective tissue. In the normal wound healing process, proteases break down damaged ECM proteins and foreign material so that new tissue can form and wound closure can occur. Excessive wound proteases lead to the breakdown of newly formed ECM and other proteins, and as a result wound healing is impaired due to damage to the ECM and abnormal prolongation of the inflammatory stage.

The present disclosure is directed to wound dressings that include a β-glucan, which may stimulate an immunomodulatory effect on cells within a wound, increase the healing rate of a wound, stimulate the healing of recalcitrant wounds (or a combination of any two or more thereof) upon application and/or over time. The wound dressings of the present technology advantageously exhibit one or more of decreased protease levels, increased growth factors levels, increased cytokine levels, protects cytokines, and protects growth factors upon application to a wound via the mixture of a 0-glucan, a collagen, and a polysaccharide. Compare with Cullen, B. et al. “Mechanism of action of PROMOGRAN, a protease modulating matrix, for the treatment of diabetic foot ulcers” Wound Rep. Reg. 2002, 10, 16-25; Cullen, B. et al. “The role of oxidised regenerated cellulose/collagen in chronic wound repair and its potential mechanism of action” Int. J. Biochem. Cell Biol. 2002, 34, 1544-1556; Hart, J. et al. “The role of oxidised regenerated cellulose/collagen in wound repair: effects in vitro on fibroblast biology and in vivo in a model of compromised healing” Int. J. Biochem. Cell Biol. 2002, 34, 1557-1570.

Thus, in an aspect, a wound dressing is provided that includes about 30 wt. % to about 95 wt. % of a collagen, about 30 wt. % to about 70 wt. % of a polysaccharide, and about 0.05 wt. % to about 10 wt. % of a β-glucan.

The β-glucan of the wound dressing may be any β-glucan, such as a β-glucan that activates macrophages. Thus, the β-glucan may include a β-glucan isolated from any source, such as sources that provide a β-glucan that activates macrophages to produce cytokines and/or growth factors. In any embodiment disclosed herein, the β-glucan may include one or more of a β-glucan isolated from a seaweed, Saccharomyces cerevisiae, Alicaligenes faecalis, Auricularia auricula-judae, Auricularia polytricha, Avena sativa, Candida utilis, Cladesporium fulvum, Claviceps purpurea, Cochiliobolus sativus, Coriolus versicolor, Corlinellus shiitake, Corticium vagum, Grifola umbellate, Hordeum vulgare, Pichia fermentans, Poria cocos, Sclerotium coffeicolum, Sclerotium delphnii, Sclerotium glucanium, Sclerotium rolsfi, Secale cereal, Shizophyllum commune, Streptococcus salvarius, Stereum sanguinolentum, Wingea robertsii, Lentines edodes, Sclerotium rolfsii, Acremonium diospyri, Monilinia fructigena, Aspergillus oryzae, Pestalotiopsis sp., Triticum sp., euglena cells, or a combination of any two or more thereof. See U.S. Pat. No. 4,946,450, incorporated herein by reference.

As discussed above, the wound dressing includes about 0.05 wt. % to about 10 wt. % of a β-glucan. Thus, the β-glucan may be included in the wound dressing in an amount of about 0.05 wt. %, about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.22 wt. %, about 0.24 wt. %, about 0.26 wt. %, about 0.28 wt. %, about 0.3 wt. %, about 0.32 wt. %, about 0.34 wt. %, about 0.36 wt. %, about 0.38 wt. %, about 0.4 wt. %, about 0.42 wt. %, about 0.44 wt. %, about 0.46 wt. %, about 0.48 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, about 5.2 wt. %, about 5.4 wt. %, about 5.6 wt. %, about 5.8 wt. %, about 6 wt. %, about 6.2 wt. %, about 6.4 wt. %, about 6.6 wt. %, about 6.8 wt. %, about 7 wt. %, about 7.2 wt. %, about 7.4 wt. %, about 7.6 wt. %, about 7.8 wt. %, about 8 wt. %, about 8.2 wt. %, about 8.4 wt. %, about 8.6 wt. %, about 8.8 wt. %, about 9 wt. %, about 9.2 wt. %, about 9.4 wt. %, about 9.6 wt. %, about 9.8 wt. %, about 10 wt. %, or any range including and/or in between any two of the preceding values.

In any embodiment disclosed herein, the collagen may be recombinant or naturally occurring. Additionally or alternatively, in some embodiments, the collagen may be a mammalian collagen, an avian collagen, a reptilian collagen, an amphibian collagen, or any combination thereof. Additionally or alternatively, in some embodiments, the collagen may be mammalian collagen. Additionally or alternatively, in some embodiments, the mammalian collagen may be a bovine collagen, a human collagen, or a combination of any two or more thereof. Additionally or alternatively, in some embodiments, the collagen may be a human collagen. Additionally or alternatively, in some embodiments, the human collagen may be human collagen type I, human collagen type III, or any combination thereof. Additionally or alternatively, in some embodiments, the collagen may be a bovine collagen. Additionally or alternatively, in some embodiments, the collagen may be bovine collagen type I, bovine collagen type II, bovine collagen type III, bovine collagen type IV, or any combination thereof. Additionally or alternatively, in some embodiments, the collagen may be bovine collagen type I and bovine collagen type III. Additionally or alternatively, in some embodiments, the collagen may be type I human recombinant collagen, type III human recombinant collagen, type I bovine collagen, type II bovine collagen, or a combination of any two or more thereof.

In any embodiment disclosed herein, the collagen may be provided by any manner known in the art. Additionally or alternatively, in some embodiments, the collagen may be provided by a tissue sample or recombinantly manufactured. Additionally or alternatively, in some embodiments, mammalian recombinant collagen may be provided by any suitable method known in the art. Additionally or alternatively, in some embodiments, human recombinant collagen may be provided by any suitable method known in the art. For example, the step of providing human recombinant collagen may comprise following the protocol described in U.S. Pat. No. 5,962,648, the entire content of which is incorporated herein by reference. Further recombinant processes are set forth in U.S. Pat. No. 5,593,859 and WO2004/078120, which are also incorporated herein by reference. Additionally or alternatively, in some embodiments, collagen will be recombinantly manufactured by culturing a cell which has been transfected with at least one gene encoding a polypeptide comprising collagen and genes encoding oxidized cellulose and subunits of the post-translational enzyme prolyl 4-hydroxylase, and purifying the resultant collagen monomer therefrom. Additionally or alternatively, in some embodiments, collagen will be recombinantly manufactured by a plant (e.g., CollPlant, CollPlant Holdings Ltd., Ness Ziona, Israel) such as tobacco, or in yeast. The human recombinant collagen solution may be subsequently subjected to polymerization or cross-linking conditions to produce an insoluble fibrous collagen.

In any embodiment disclosed herein, the collagen may be a type I collagen, a type II collagen, or a type III collagen. Additionally or alternatively, in some embodiments, the collagen may be obtained from any natural source, may be chemically-modified collagen (e.g., an atelocollagen obtained by removing the immunogenic telopeptides from natural collagen), or may be any combination thereof. For example, the collagen may include collagen obtained from bovine corium that has been rendered largely free of non-collagenous components, for example, including fat, non-collagenous proteins, polysaccharides, and other carbohydrates, such as by procedures described in U.S. Pat. Nos. 4,614,794 and 4,320,201, the entire contents of which are incorporated by reference.

The wound dressing includes about 30 wt. % to about 95 wt. % of a collagen; thus, the collagen may be included in the wound dressing in an amount of about 30 wt. %, about 32 wt. %, about 34 wt. %, about 36 wt. %, about 38 wt. %, about 40 wt. %, about 42 wt. %, about 44 wt. %, about 46 wt. %, about 48 wt. %, about 50 wt. %, about 52 wt. %, about 54 wt. %, about 56 wt. %, about 58 wt. %, about 60 wt. %, about 62 wt. %, about 64 wt. %, about 66 wt. %, about 68 wt. %, about 70 wt. %, about 72 wt. %, about 74 wt. %, about 76 wt. %, about 78 wt. %, about 80 wt. %, about 82 wt. %, about 84 wt. %, about 86 wt. %, about 88 wt. %, about 90 wt. %, about 92 wt. %, about 94 wt. %, about 95 wt. %, or any range including and/or in between any two of the preceding values.

In any embodiment disclosed herein, the collagen may have a weight-average molecular weight of about 5,000 to about 100,000. Additionally or alternatively, in some embodiments, the collagen may have a weight-average molecular weight of about 5,000, about 6,000, about 7,000, about 8,000, about 9,000, about 10,000, about 11,000, about 12,000, about 13,000, about 14,000, about 15,000, about 16,000, about 17,000, about 18,000, about 19,000, about 20,000, about 22,000, about 24,000, about 26,000, about 28,000, about 30,000, about 32,000, about 34,000, about 36,000, about 38,000, about 40,000, about 42,000, about 44,000, about 46,000, about 48,000, about 50,000, about 52,000, about 54,000, about 56,000, about 58,000, about 60,000, about 62,000, about 64,000, about 66,000, about 68,000, about 70,000, about 72,000, about 74,000, about 76,000, about 78,000, about 80,000, about 82,000, about 84,000, about 86,000, about 88,000, about 90,000, about 92,000, about 94,000, about 96,000, about 98,000, about 100,000, or any range including and/or in between any two of the preceding values.

In any embodiment disclosed herein, the collagen of the wound dressing may include a weight ratio of human collagen type I to human collagen type III of about 100:0, about 90:10, about 80:20, about 70:30, about 60:40, about 50:50, about 40:60, about 30:70, about 20:80, about 10:90, about 0:100, or any range including and/or in between any two of these values. Additionally or alternatively, in some embodiments, the ratio by weight of human collagen type I to human collagen type III is greater than about 50:50, or greater than about 70:30. Additionally or alternatively, in some embodiments, the collagen of the wound dressing may include a weight ratio of type I bovine collagen to type III bovine collagen of about 85:15.

In any embodiment disclosed herein, the polysaccharide may comprise oxidized regenerated cellulose (ORC), chitin, chitosan, an alginate, methyl cellulose, carboxymethyl cellulose, a glycosaminoglycan, chondroitin sulfate, heparin sulfate, or a combination of any two or more thereof. Additionally or alternatively, in some embodiments, the polysaccharide is oxidized regenerated cellulose (ORC).

In any embodiment disclosed herein, the ORC may have a weight-average molecular weight of about 50,000 to about 1,000,000. Additionally or alternatively, in some embodiments, the ORC may have a weight-average molecular weight of about 50,000, about 55,000, about 60,000, about 65,000, about 70,000, about 75,000, about 80,000, about 85,000, about 90,000, about 95,000, about 100,000, about 110,000, about 120,000, about 130,000, about 140,000, about 150,000, about 160,000, about 170,000, about 180,000, about 190,000, about 200,000, about 210,000, about 220,000, about 230,000, about 240,000, about 250,000, about 260,000, about 270,000, about 280,000, about 290,000, about 300,000, about 310,000, about 320,000, about 330,000, about 340,000, about 350,000, about 360,000, about 370,000, about 380,000, about 390,000, about 400,000, about 410,000, about 420,000, about 430,000, about 440,000, about 450,000, about 460,000, about 470,000, about 480,000, about 490,000, about 500,000, about 510,000, about 520,000, about 530,000, about 540,000, about 550,000, about 560,000, about 570,000, about 580,000, about 590,000, about 600,000, about 610,000, about 620,000, about 630,000, about 640,000, about 650,000, about 660,000, about 670,000, about 680,000, about 690,000, about 700,000, about 710,000, about 720,000, about 730,000, about 740,000, about 750,000, about 760,000, about 770,000, about 780,000, about 790,000, about 800,000, about 810,000, about 820,000, about 830,000, about 840,000, about 850,000, about 860,000, about 870,000, about 880,000, about 890,000, about 900,000, about 910,000, about 920,000, about 930,000, about 940,000, about 950,000, about 960,000, about 970,000, about 980,000, about 990,000, about 1,000,000, or any range including and/or in between any two of the preceding values.

In any embodiment disclosed herein, oxidized regenerated cellulose (ORC) may be produced by the oxidation of cellulose, for example with dinitrogen tetroxide and/or as described in U.S. Pat. No. 3,122,479 (incorporated herein by reference). Without wishing to be bound by theory, it is believed that this process may convert primary alcohol groups on the saccharide residues of the cellulose to carboxylic acid groups, for example, forming uronic acid residues within the cellulose chain. The oxidation may not proceed with complete selectivity, and as a result hydroxyl groups on carbons 2 and 3 of the saccharide residue may be converted to the keto form. These ketone units may introduce an alkali labile link, which at pH 7 or higher initiates the decomposition of the polymer via formation of a lactone and sugar ring cleavage. As a result, oxidized regenerated cellulose is biodegradable and bioresorbable under physiological conditions. ORC is available with a variety of degrees of oxidation and hence rates of degradation. The ORC may include particles, fibers, or both; in any embodiment disclosed herein, the ORC may be in the form of particles, such as fiber particles or powder particles.

As discussed above, the wound dressing includes from about 30 wt. % to about 70 wt. % of the polysaccharide. Thus, the polysaccharide may be included in the wound dressing in an amount of about 30 wt. %, about 32 wt. %, about 34 wt. %, about 36 wt. %, about 38 wt. %, about 40 wt. %, about 42 wt. %, about 44 wt. %, about 46 wt. %, about 48 wt. %, about 50 wt. %, about 52 wt. %, about 54 wt. %, about 56 wt. %, about 58 wt. %, about 60 wt. %, about 62 wt. %, about 64 wt. %, about 66 wt. %, about 68 wt. %, about 70 wt. %, or any range including and/or in between any two of these values.

The wound dressing of any embodiment disclosed herein may include a weight ratio of the collagen to the polysaccharide of about 60:40 to about 40:60. Thus, the wound dressing composition may include a weight ratio of the collagen to the polysaccharide of about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, or any range including and/or in between any two of these values. Additionally or alternatively, in some embodiments, in some embodiments, the weight ratio of the collagen to the polysaccharide may be about 55:45.

In any embodiment disclosed herein, the wound dressing may have a thickness of about 0.5 cm to about 5 cm. Thus, the wound dressing may have a thickness of about 0.5 cm, about 0.6 cm, about 0.7 cm, about 0.8 cm, about 0.9 cm, about 1 cm, about 1.1 cm, about 1.2 cm, about 1.3 cm, about 1.4 cm, about 1.5 cm, about 1.6 cm, about 1.7 cm, about 1.8 cm, about 1.9 cm, about 2 cm, about 2.1 cm, about 2.2 cm, about 2.3 cm, about 2.4 cm, about 2.5 cm, about 2.6 cm, about 2.7 cm, about 2.8 cm, about 2.9 cm, about 3 cm, about 3.1 cm, about 3.2 cm, about 3.3 cm, about 3.4 cm, about 3.5 cm, about 3.6 cm, about 3.7 cm, about 3.8 cm, about 3.9 cm, about 4 cm, about 4.1 cm, about 4.2 cm, about 4.3 cm, about 4.4 cm, about 4.5 cm, about 4.6 cm, about 4.7 cm, about 4.8 cm, about 4.9 cm, about 5 cm, or any range including and/or in between any two of these values.

In any embodiment disclosed herein, the wound dressing may include a silver compound.

Additionally or alternatively, in some embodiments, the wound dressing may include about 0.1 wt. % to about 3 wt. % of the silver compound. Thus, the silver compound may be included in the wound dressing composition in an amount of about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.22 wt. %, about 0.24 wt. %, about 0.25 wt. %, about 0.26 wt. %, about 0.28 wt. %, about 0.3 wt. %, about 0.32 wt. %, about 0.34 wt. %, about 0.36 wt. %, about 0.38 wt. %, about 0.4 wt. %, about 0.42 wt. %, about 0.44 wt. %, about 0.46 wt. %, about 0.48 wt. %, about 0.50 wt. %, about 0.52 wt. %, about 0.54 wt. %, about 0.56 wt. %, about 0.58 wt. %, about 0.6 wt. %, about 0.62 wt. %, about 0.64 wt. %, about 0.66 wt. %, about 0.68 wt. %, about 0.7 wt. %, about 0.72 wt. %, about 0.74 wt. %, about 0.76 wt. %, about 0.78 wt. %, about 0.8 wt. %, about 0.82 wt. %, about 0.84 wt. %, about 0.86 wt. %, about 0.88 wt. %, about 0.9 wt. %, about 0.92 wt. %, about 0.94 wt. %, about 0.96 wt. %, about 0.98 wt. %, about 1 wt. %, about 1.1 wt. %, about 1.15 wt. %, about 1.2 wt. %, about 1.25 wt. %, about 1.3 wt. %, about 1.35 wt. %, about 1.4 wt. %, about 1.45 wt. %, about 1.5 wt. %, about 1.55 wt. %, about 1.6 wt. %, about 1.65 wt. %, about 1.7 wt. %, about 1.75 wt. %, about 1.8 wt. %, about 1.85 wt. %, about 1.9 wt. %, about 1.95 wt. %, about 2 wt. %, about 2.05 wt. %, about 2.1 wt. %, about 2.15 wt. %, about 2.2 wt. %, about 2.25 wt. %, about 2.3 wt. %, about 2.35 wt. %, about 2.4 wt. %, about 2.45 wt. %, about 2.5 wt. %, about 2.55 wt. %, about 2.6 wt. %, about 2.65 wt. %, about 2.7 wt. %, about 2.75 wt. %, about 2.8 wt. %, about 2.85 wt. %, about 2.9 wt. %, about 2.95 wt. %, about 3 wt. %, or any range including and/or in between any two of the preceding values.

In any embodiment disclosed herein, the silver compound may be one or more pharmaceutically acceptable salts. Additionally or alternatively, in some embodiments, the one or more pharmaceutically acceptable silver salts may include, but are not limited to, silver oxide, silver chromate, silver allantoinate, silver borate, silver glycerolate, silver nitrate, silver acetate, silver chloride, silver sulfate, silver lactate, silver bromide, silver iodide, silver carbonate, silver citrate, silver laurate, silver deoxycholate, silver salicylate, silver p-aminobenzoate, silver p-aminosalicylate, nanocrystalline silver, a silver oxysalt, or a combination of any two or more thereof.

In any embodiment disclosed herein, the wound dressing may further include one or more additional biomaterials, an antioxidant, a plasticizer, a growth factor, or a combination of any two or more thereof.

In any embodiment disclosed herein, the additional biomaterials may be selected from the group consisting of gelatin, fibronectin, and a combination of any two or more thereof.

In any embodiment disclosed herein, the wound dressing may include about 0.1 wt. % to about 10 wt. % of the additional biomaterials. Thus, the additional biomaterials may be included in the wound dressing in an amount of about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.22 wt. %, about 0.24 wt. %, about 0.26 wt. %, about 0.28 wt. %, about 0.3 wt. %, about 0.32 wt. %, about 0.34 wt. %, about 0.36 wt. %, about 0.38 wt. %, about 0.4 wt. %, about 0.42 wt. %, about 0.44 wt. %, about 0.46 wt. %, about 0.48 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, about 5.2 wt. %, about 5.4 wt. %, about 5.6 wt. %, about 5.8 wt. %, about 6 wt. %, about 6.2 wt. %, about 6.4 wt. %, about 6.6 wt. %, about 6.8 wt. %, about 7 wt. %, about 7.2 wt. %, about 7.4 wt. %, about 7.6 wt. %, about 7.8 wt. %, about 8 wt. %, about 8.2 wt. %, about 8.4 wt. %, about 8.6 wt. %, about 8.8 wt. %, about 9 wt. %, about 9.2 wt. %, about 9.4 wt. %, about 9.6 wt. %, about 9.8 wt. %, about 10 wt. %, or any range including and/or in between any two of the preceding values.

In any embodiment disclosed herein, the antioxidant may include one or more of anthocyanins, astaxanthin, bilirubin, canthaxanthin, capsaicin, citric acid, curcumin, coenzyme Q10, eugenol, flavanols, flavonolignans, flavanones, flavones, flavonols, iodide, isoflavone phytoestrogens, lutein, lycopene, manganese, melatonin, N-acetylcysteine, oxalic acid, phenolic acids, phytic acid, R-α-lipoic acid, stilbenoids, tocopherol, tocotrienol, vitamin A, vitamin C, vitamin E, xanthones, zeaxanthin, α-carotene, β-carotene, or a combination of any two or more thereof.

Additionally or alternatively, in some embodiments, the anthocyanins may include, but are not limited to, one or more of cyanidin, delphinidin, malvidin, pelargonidin, peonidin, petunidin, or a combination of any two or more thereof.

Additionally or alternatively, in some embodiments, the flavanols may include, but are not limited to, one or more of catechin, epicatechin, theaflavin, thearubigins, gallocatechin, epigallocatechin, a gallate ester of any thereof, or a combination of any two or more thereof.

Additionally or alternatively, in some embodiments, the flavanones may include, but are not limited to, one or more of eriodictyol, hesperetin, naringenin, or a combination of any two or more thereof.

Additionally or alternatively, in some embodiments, the flavones may include, but are not limited to, one or more of apigenin, luteolin, tangeritin, or a combination of any two or more thereof.

Additionally or alternatively, in some embodiments, the flavonols may include, but are not limited to, one or more of isorhamnetin, kaempferol, myricetin, proanthocyanidins, quercetin, rutin, or a combination of any two or more thereof.

Additionally or alternatively, in some embodiments, the isoflavone phytoestrogens may include, but are not limited to, one or more of daidzein, genistein, glycitein, or a combination of any two or more thereof.

Additionally or alternatively, in some embodiments, the phenolic acids may include, but are not limited to, one or more of chicoric acid, chlorogenic acid, cinnamic acid, ellagic acid, ellagitannins, gallic acid, gallotannins, rosmarinic acid, salicylic acid, or any ester thereof, or a combination of any two or more thereof.

Additionally or alternatively, in some embodiments, the stillbenoids may include, but are not limited to, one or more of resveratrol, pterostilbene, or a combination of any two or more thereof.

In any embodiment disclosed herein, the wound dressing may include about 0.1 wt. % to about 10 wt. % of the antioxidant. Thus, the antioxidant may be included in the wound dressing in an amount of about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.22 wt. %, about 0.24 wt. %, about 0.26 wt. %, about 0.28 wt. %, about 0.3 wt. %, about 0.32 wt. %, about 0.34 wt. %, about 0.36 wt. %, about 0.38 wt. %, about 0.4 wt. %, about 0.42 wt. %, about 0.44 wt. %, about 0.46 wt. %, about 0.48 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, about 5.2 wt. %, about 5.4 wt. %, about 5.6 wt. %, about 5.8 wt. %, about 6 wt. %, about 6.2 wt. %, about 6.4 wt. %, about 6.6 wt. %, about 6.8 wt. %, about 7 wt. %, about 7.2 wt. %, about 7.4 wt. %, about 7.6 wt. %, about 7.8 wt. %, about 8 wt. %, about 8.2 wt. %, about 8.4 wt. %, about 8.6 wt. %, about 8.8 wt. %, about 9 wt. %, about 9.2 wt. %, about 9.4 wt. %, about 9.6 wt. %, about 9.8 wt. %, about 10 wt. %, or any range including and/or in between any two of the preceding values.

In any embodiment disclosed herein, the plasticizer may include an acetylated monoglyceride, an alkyl citrate, methyl ricinoleate, glycerol, or a combination of any two or more thereof. Additionally or alternatively, in some embodiments, the alkyl citrate may be one or more of triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, or a combination of any two or more thereof.

In any embodiment disclosed herein, the wound dressing may include about 0.1 wt. % to about 10 wt. % of the plasticizer. Thus, the plasticizer may be included in the wound dressing in an amount of about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.22 wt. %, about 0.24 wt. %, about 0.26 wt. %, about 0.28 wt. %, about 0.3 wt. %, about 0.32 wt. %, about 0.34 wt. %, about 0.36 wt. %, about 0.38 wt. %, about 0.4 wt. %, about 0.42 wt. %, about 0.44 wt. %, about 0.46 wt. %, about 0.48 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, about 5.2 wt. %, about 5.4 wt. %, about 5.6 wt. %, about 5.8 wt. %, about 6 wt. %, about 6.2 wt. %, about 6.4 wt. %, about 6.6 wt. %, about 6.8 wt. %, about 7 wt. %, about 7.2 wt. %, about 7.4 wt. %, about 7.6 wt. %, about 7.8 wt. %, about 8 wt. %, about 8.2 wt. %, about 8.4 wt. %, about 8.6 wt. %, about 8.8 wt. %, about 9 wt. %, about 9.2 wt. %, about 9.4 wt. %, about 9.6 wt. %, about 9.8 wt. %, about 10 wt. %, or any range including and/or in between any two of the preceding values.

In any embodiment disclosed herein, the growth factor may include one or more of an epidermal growth factor (EGF), a hepatocyte growth factor (HGF), a fibroblast growth factor (FGF), a platelet-derived growth factor (PDGF), or a combination of any two or more thereof. Additionally or alternatively, in some embodiments, the fibroblast growth factor may include one or more of fibroblast growth factor 1 (FGF1), fibroblast growth factor 2 (FGF2), fibroblast growth factor 3 (FGF3), fibroblast growth factor 4 (FGF4), fibroblast growth factor 5 (FGF5), fibroblast growth factor 6 (FGF6), fibroblast growth factor 7/keratinocyte growth factor (FGF7/KGF), fibroblast growth factor 8 (FGF8), fibroblast growth factor 9 (FGF9), fibroblast growth factor 10/keratinocyte growth factor 2 (FGF10/KGF2), fibroblast growth factor 11 (FGF11), fibroblast growth factor 12 (FGF12), fibroblast growth factor 13 (FGF13), fibroblast growth factor 14 (FGF14), fibroblast growth factor 15 (FGF15), fibroblast growth factor 16 (FGF16), fibroblast growth factor 17 (FGF17), fibroblast growth factor 18 (FGF18), fibroblast growth factor 19 (FGF19), fibroblast growth factor 20 (FGF20), fibroblast growth factor 21 (FGF21), fibroblast growth factor 22 (FGF22), fibroblast growth factor 23 (FGF23), or a combination of any two or more thereof.

In any embodiment disclosed herein, the wound dressing may include about 0.1 wt. % to about 10 wt. % of the growth factor. Thus, the growth factor may be included in the wound dressing in an amount of about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.22 wt. %, about 0.24 wt. %, about 0.26 wt. %, about 0.28 wt. %, about 0.3 wt. %, about 0.32 wt. %, about 0.34 wt. %, about 0.36 wt. %, about 0.38 wt. %, about 0.4 wt. %, about 0.42 wt. %, about 0.44 wt. %, about 0.46 wt. %, about 0.48 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, about 5.2 wt. %, about 5.4 wt. %, about 5.6 wt. %, about 5.8 wt. %, about 6 wt. %, about 6.2 wt. %, about 6.4 wt. %, about 6.6 wt. %, about 6.8 wt. %, about 7 wt. %, about 7.2 wt. %, about 7.4 wt. %, about 7.6 wt. %, about 7.8 wt. %, about 8 wt. %, about 8.2 wt. %, about 8.4 wt. %, about 8.6 wt. %, about 8.8 wt. %, about 9 wt. %, about 9.2 wt. %, about 9.4 wt. %, about 9.6 wt. %, about 9.8 wt. %, about 10 wt. %, or any range including and/or in between any two of the preceding values.

In any embodiment disclosed herein, the wound dressing composition of the present disclosure may be sterile and packaged in a microorganism-impermeable container.

Based on the description of the wound dressing of the present technology and the knowledge of a person of ordinary skill in the art (as illustrated in U.S. Pat. No. 6,309,454, incorporated herein by reference), a person of ordinary skill in the art will understand how to generate compositions of the present technology.

### Negative-Pressure Therapy

The wound dressing of any embodiment described herein may be employed in therapy in which a wound is treated with reduced pressure. Treatment of a wound with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and/or micro-deformation of tissue at a wound site. Together, these benefits may increase development of granulation tissue and reduce healing times.

Generally, the system may be configured to provide negative-pressure to a wound in accordance with this specification. In any embodiment herein, the system may generally include a negative-pressure supply, and may include or be configured to be coupled to a distribution component. In general, a distribution component may refer to any complementary or ancillary component configured to be fluidly coupled to a negative-pressure supply in a fluid path between a negative-pressure supply and a wound.

In any embodiment herein, the wound dressing may be configured to distribute negative pressure. Additionally or alternatively, the fluid path(s) may be reversed or a secondary fluid path may be provided to facilitate movement of fluid across a wound. Additionally or alternatively, the fluid pathways of the through-holes may be interconnected to improve distribution or collection of fluids.

The fluid mechanics associated with using a negative-pressure source to reduce pressure in another component or location, such as within a sealed therapeutic environment, can be mathematically complex. However, the basic principles of fluid mechanics applicable to negative-pressure therapy are generally well-known to those skilled in the art. The process of reducing pressure may be described generally and illustratively herein as “delivering,” “distributing,” or “generating” negative pressure, for example.

In general, a fluid, such as wound fluid (for example, wound exudates and other fluids), flows toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. This orientation is generally presumed for purposes of describing various features and components herein. However, the fluid path may also be reversed in some applications (such as by substituting a positive-pressure source for a negative-pressure source) and this descriptive convention should not be construed as a limiting convention.

“Negative pressure” may generally refer to a pressure less than a local ambient pressure, such as the ambient pressure in a local environment external to a sealed therapeutic environment provided by the wound dressing. In many cases, the local ambient pressure may also be the atmospheric pressure proximate to or about a wound. Alternatively or additionally, the pressure may be less than a hydrostatic pressure associated with the tissue at the wound. While the amount and nature of negative pressure applied to a wound may vary according to therapeutic requirements, the pressure is generally a low vacuum, also commonly referred to as a rough vacuum, between −5 mm Hg (−667 Pa) and −500 mm Hg (−66.7 kPa), gauge pressure. Common therapeutic ranges are between −50 mm Hg (−6.7 kPa) and −300 mm Hg (−39.9 kPa), gauge pressure.

Additionally or alternatively, in any embodiment herein, a negative-pressure supply may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components, such as sensors, processing units, alarm indicators, memory, databases, software, display devices, or user interfaces that further facilitate therapy. A negative-pressure source may be combined with a controller and other components into a therapy unit. A negative-pressure supply may also have one or more supply ports configured to facilitate coupling and de-coupling of the negative-pressure supply to one or more distribution components.

In any embodiment herein, components may be fluidly coupled to each other to provide a path for transferring fluids (i.e., liquid and/or gas) between the components. For example, components may be fluidly coupled through a fluid conductor, such as a tube. As used herein, the term “fluid conductor” may include a tube, pipe, hose, conduit, or other structure with one or more lumina or open passages adapted to convey a fluid between two ends thereof. Typically, a fluid conductor may be an elongated, cylindrical structure with some flexibility, but the geometry and rigidity may vary. Additionally or alternatively, in any embodiment herein, the negative-pressure source may be operatively coupled to the wound dressing via an interface.

### Treatment Methods of the Present Technology

In an aspect, methods for treating a wound in a subject in need thereof are provided, wherein the method includes administering to the wound a wound dressing of any embodiment disclosed herein. The wound may be an acute wound or a chronic wound. Exemplary chronic wounds include, but are not limited to, infectious wounds, venous ulcers, decubitis ulcers, and/or diabetic ulcers; exemplary acute wounds include, but are not limited to, surgical wounds, trauma wounds, burn wounds, and/or donor sites. In any embodiment disclosed herein, the subject is human.

Additionally or alternatively, in some embodiments, the wound dressing may be administered directly to the wound. Any method known to those in the art for administering a wound dressing to an acute or a chronic wound disclosed herein may be employed. Suitable methods include in vitro or in vivo methods. In vivo methods typically include the administration of one or more wound dressings to a subject in need thereof, suitably a human. When used in vivo for therapy, the one or more wound dressings described herein are administered to the subject in effective amounts (i.e., amounts that have desired therapeutic effect). The dose and dosage regimen will depend upon the state of the wound of the subject, and the characteristics of the particular wound dressing used.

The effective amount may be determined during pre-clinical trials and clinical trials by methods familiar to physicians and clinicians. An effective amount of one or more wound dressings useful in the methods may be administered to a subject in need thereof by any number of well-known methods for administering wound dressings.

In any embodiment disclosed herein, the wound dressings may be administered daily for 1 hour or more, for 2 hours or more, for 3 hours or more, for 4 hours or more, for 5 hours or more, for 6 hours or more, for 12 hours or more. In any embodiment disclosed herein, the wound dressings may be administered one, two, three, four, or five times per day. In any embodiment disclosed herein, the wound dressings may be administered daily for one, two, three, four or five weeks. In any embodiment disclosed herein, the wound dressings may be administered daily for less than 6 weeks. In any embodiment disclosed herein, the wound dressings may be administered daily for 6 weeks or more. In any embodiment disclosed herein, the wound dressings may be administered daily for 12 weeks or more. In any embodiment disclosed herein, the wound dressings may be administered every day, every other day, every third day, every fourth day, every fifth day, or every sixth day. In any embodiment disclosed herein, the wound dressings may be administered weekly, bi-weekly, tri-weekly, or monthly. In any embodiment disclosed herein, the wound dressings may be administered for a period of one, two, three, four, or five weeks. In any embodiment disclosed herein, the wound dressings may be administered for six weeks or more. In any embodiment disclosed herein, the wound dressings may be administered for twelve weeks or more. In any embodiment disclosed herein, the wound dressings may be administered for a period of less than one year. In any embodiment disclosed herein, the wound dressings may be administered for a period of more than one year. In any embodiment disclosed herein, the wound dressings may be administered for a chronic wound as appropriate.

In any embodiment herein, the method may include employing the wound dressing in the context of a negative-pressure therapy, where the negative-pressure therapy may include positioning the wound dressing in and/or proximate to the wound. The negative-pressure therapy may further include sealing the wound dressing to tissue surrounding the wound to form a sealed space. For example, the wound dressing may be positioned in and/or proximate to the wound and sealed to an attachment surface near the wound, for example, to undamaged epidermis peripheral to a wound.

The negative-pressure therapy method in any embodiment herein may further include fluidly coupling a negative-pressure source to the sealed space and operating the negative-pressure source to generate a negative pressure in the sealed space. For example, the negative-pressure source may be coupled to the wound dressing such that the negative-pressure source may be used to reduce the pressure in the sealed space. For example, negative pressure applied across the wound, for example, via the wound dressing may be effective to induce macrostrain and microstrain at the wound site, as well as remove exudates and other fluids from the wound.

In any embodiment herein, it may be the method increases one or more growth factors in the wound. In any embodiment herein, it may be the method increases one or more cytokines in the wound. In any embodiment herein, it may be the method decreases protease activity in the wound. In any embodiment herein, it may be the method induces growth factor production at the site of the wound. In any embodiment herein, it may be the method induces cytokine production at the site of the wound. In any embodiment herein, it may be the method decreases inflammation at the site of the wound. In any embodiment herein, it may be the method increases the rate of healing of the wound (e.g., an increase greater than using a wound dressing with all the same components as a wound dressing of the present technology but absent a β-glucan).

### Kits Comprising the Wound Dressing of the Present Technology

In a further related aspect, the present disclosure provides kits that include a wound dressing of any embodiment described herein and instructions for use. The kits of the present technology may also include instructions for methods for treating a wound in a subject in need thereof. The kit may optionally comprise components such as antiseptic wipes, ointment, adhesive tape, tweezers, scissors, etc.

In a further related aspect, the present disclosure provides a kit that includes a dressing and instructions for making a wound dressing of any embodiment disclosed herein, where the dressing includes about 30 wt. % to about 95 wt. % of a collagen; and about 30 wt. % to about 70 wt. % of a polysaccharide. Thus, the dressing may have the same components as a wound dressing of any embodiment disclosed herein but without the β-glucan of the wound dressings of the present technology. The instructions for making the wound dressing may include description of a method for addition of a β-glucan (of any embodiment described herein) to the dressing, such as by applying a solution that includes the β-glucan (e.g., spraying, coating, or both) to the dressing.

### EXAMPLES

The present technology is further illustrated by the following examples, which should not be construed as limiting in any way.

### Example 1: Exemplary Wound Dressings of the Present Technology

β-glucan powder (isolated from S. cerevisiae; Sigma, Product code 1048288) was dissolved in 5 mL of water and added to a slurry containing 55% bovine collagen, 45% oxidized regenerated cellulose, 0.05M acetic acid and with a solids content of 1%. Three different exemplary slurries were generated with differing amounts of the β-glucan:

(a) 1.3 mg β-glucan powder to 200 g slurry;

(b) 10.5 mg β-glucan powder to 200 g slurry; and

(c) 26.2 mg β-glucan powder to 200 g slurry.

Each slurry (a)-(c) was then blended three times at 7 second bursts to avoid heating and denaturation and degassed under vacuum for 10 minutes to remove all bubbles. Samples of each slurry were then weighed out for the desired dressing thickness and freeze-dried to form a wound dressing of the present technology, with the following amounts of β-glucan in the respective wound dressing:

(a) 0.05 wt. %;

(b) 0.4 wt. %; and

(c) 1 wt. %.

### Example 2: Wound Dressing Compositions of the Present Technology Decrease Protease Activity In Vitro

A fluorometric assay will be utilized to determine residual protease activities after incubation with the test wound dressing samples. Briefly, 6 mm punch biopsy samples of the wound dressings of the present technology, a standard collagen/ORC dressing (e.g., PROMOGRAN™ Matrix) or a conventional wound dressing (e.g., SOF-WICK™) are pre-wet with phosphate buffered saline (PBS). The wet dressing samples are then incubated at 37° C. either in a solution (60 μL) of simulated wound fluid (SWF) containing human neutrophil elastase (HNE, 273 mU/mL), or matrix metalloproteinase-9 (MMP-9, 1 μg/mL). Both solutions further include 2% bovine serum albumin (BSA). After incubation at 37° C., 5 μL aliquots are taken from each solution, in duplicate, at 0.5, 1, 2, and 24 hours.

Dressing treated SWF samples are then evaluated by fluorometric enzymatic activity assay to quantify residual protease activities. For the evaluation of the residual activity of HNE in the SWF, aliquots are transferred to activity assay buffer (0.1 M HEPES, 0.5 M NaCl, pH 7.5, 10% DMSO) and are incubated in the presence of the appropriate peptide substrate (MeOSuc-Ala-Ala-Pro-Val-AMC). For evaluation of the residual activity of MMP-9 in the SWF, aliquots are transferred to activity assay buffer (40 mM Tris-HCl, 200 mM NaCl, 10 mM CaCl₂), pH 7.4) and are incubated in the presence of the appropriate peptide substrate (Suc-Gly-Leu-Gly-Pro-AMC). Upon incubation, fluorescence is monitored for each sample with respect to time with an Ex/Em (nm) of 383/455. Results are expressed as a percentage of the initial enzyme activity at time 0 hours. It is anticipated that the wound dressings of the present technology will exhibit reduced protease activity levels and improved wound healing of chronic or acute wounds compared to standard collagen/ORC dressings (e.g., PROMOGRAN™ Matrix) and/or conventional wound dressings (e.g., SOF-WICK™).

Accordingly, the wound dressing compositions of the present technology are useful in methods for treating a wound in a subject in need thereof, wherein the method comprises administering to the wound a dressing of any embodiment disclosed herein.

### Example 3: Wound Dressing Compositions of the Present Technology Decrease Protease Activity in a Wound Upon Application

A fluorometric assay will be utilized to determine residual protease activities after incubation with the test wound dressing samples. Briefly, 6 mm punch biopsy samples of the wound dressings of the present technology, a standard collagen/ORC dressing (e.g., PROMOGRAN™ Matrix) or a conventional wound dressing (e.g., SOF-WICK™) are pre-wet with phosphate buffered saline (PBS). The wet dressing samples are then incubated at 37° C. either in a solution (60 μL) of wound fluid collected from a diabetic foot ulcer (DFU), a venous leg ulcer (VLU), or a pressure ulcer (PU) containing human neutrophil elastase (HNE, 273 mU/mL of wound fluid), or matrix metalloproteinase-9 (MMP-9, 1 μg/mL of wound fluid). After incubation at 37° C., 5 μL aliquots are taken from each solution, in duplicate, at 0.5, 1, 2, and 24 hours.

Dressing treated wound fluid samples are then evaluated by fluorometric enzymatic activity assay to quantify residual protease activities. For the evaluation of the residual activity of HNE in the DFU, VLU, or PU samples, aliquots are transferred to activity assay buffer (0.1 M HEPES, 0.5 M NaCl, pH 7.5, 10% DMSO) and are incubated in the presence of the appropriate peptide substrate (MeOSuc-Ala-Ala-Pro-Val-AMC). For evaluation of the residual activity of MMP-9 in the DFU, VLU, or PU samples, aliquots are transferred to activity assay buffer (40 mM Tris-HCl, 200 mM NaCl, 10 mM CaCl₂, pH 7.4) and are incubated in the presence of the appropriate peptide substrate (Suc-Gly-Leu-Gly-Pro-AMC). Upon incubation, fluorescence is monitored for each sample with respect to time with an Ex/Em (nm) of 383/455. Results are expressed as a percentage of the initial enzyme activity at time 0 hours. It is anticipated that the wound dressings of the present technology will exhibit reduced protease activity levels and improved wound healing of chronic or acute wounds compared to standard collagen/ORC dressings (e.g., PROMOGRAN™ Matrix) and/or conventional wound dressings (e.g., SOF-WICK™).

Accordingly, the wound dressing compositions of the present technology are useful in methods for treating a wound in a subject in need thereof, wherein the method comprises administering to the wound a dressing of any embodiment disclosed herein.

### Example 4: Wound Dressing Compositions of the Present Technology Increase Growth Binding and Protection In Vitro

An enzyme-linked immunosorbent assay (ELISA) assay will be utilized to determine the binding and protection of growth factors in SWF after incubation with the test wound dressing samples. Briefly, 6 mm punch biopsy samples of the wound dressings of the present technology, a standard collagen/ORC dressing (e.g., PROMOGRAN™ Matrix) or a conventional wound dressing (e.g., SOF-WICK™) are pre-wet with phosphate buffered saline (PBS). The wet dressing samples are then transferred to a 100 μL solution containing either PDGF-BB (100 ng/mL), EGF (1 ng/ML), or VEGF (1 ng/ML), and further containing a proteolytic enzyme (e.g., HNE, 25 μg/mL). The samples are then incubated at 37° C. overnight (about 16 to about 18 hours). Following incubation, the dressing biopsies will be removed from the solution and washed with 1.0 M NaCl to recover the bound growth factor. The amount of bound/recovered growth factor and unbound growth factor remaining in solution will be determined by ELISA.

It is anticipated that the wound dressings of the present technology will exhibit increased protection and recovery of growth factors, and further improved wound healing of chronic or acute wounds compared to standard collagen/ORC dressings (e.g., PROMOGRAN™ Matrix) and/or conventional wound dressings (e.g., SOF-WICK™).

Accordingly, the wound dressing compositions of the present technology are useful in methods for treating a wound in a subject in need thereof, wherein the method comprises administering to the wound a dressing of any embodiment disclosed herein.

### Example 5: Wound Dressing Compositions of the Present Technology Increase Growth Binding and Protection in a Wound Upon Application

An enzyme-linked immunosorbent assay (ELISA) assay will be utilized to determine the binding and protection of growth factors in SWF after incubation with the test wound dressing samples. Briefly, 6 mm punch biopsy samples of the wound dressings of the present technology, a standard collagen/ORC dressing (e.g., PROMOGRAN™ Matrix) or a conventional wound dressing (e.g., SOF-WICK™) are pre-wet with phosphate buffered saline (PBS). The wet dressing samples are then transferred to a 100 μL solution of wound fluid collected from a diabetic foot ulcer (DFU), a venous leg ulcer (VLU), or a pressure ulcer (PU). The samples are then incubated at 37° C. overnight (about 16 to about 18 hours). Following incubation, the dressing biopsies will be removed from the solution and washed with 1.0 M NaCl to recover the bound growth factor. The amount of bound/recovered growth factors and unbound growth factors (e.g., PDGF-AA, PDGF-AB, and PDGF-BB) remaining in solution will be determined by ELISA.

It is anticipated that the wound dressings of the present technology will exhibit increased protection and recovery of growth factors (e.g., PDGF-AA, PDGF-AB, and PDGF-BB), and further improved wound healing of chronic or acute wounds compared to standard collagen/ORC dressings (e.g., PROMOGRAN™ Matrix) and/or conventional wound dressings (e.g., SOF-WICK™).

Accordingly, the wound dressing compositions of the present technology are useful in methods for treating a wound in a subject in need thereof, wherein the method comprises administering to the wound a dressing of any embodiment disclosed herein.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions, or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third, and upper third, etc. As will also be understood by one skilled in the art all language such as “up to,” “at least,” “greater than,” “less than,” “at most,” “down to,” and the like, include the number recited and refer to ranges which can be subsequently broken down into subranges as discussed above. As will also be understood by one skilled in the art all language such as “greater than,” “less than,” and the like, refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A wound dressing comprising
about 30 wt. % to about 95 wt. % of a collagen;
about 30 wt. % to about 70 wt. % of a polysaccharide; and
about 0.05 wt. % to about 10 wt. % of a β-glucan.

2. The wound dressing of claim 1, where the β-glucan comprises one or more of a β-glucan isolated from Saccharomyces cerevisiae, Alicaligenes faecalis, Auricularia auricula-judae, Auricularia polytricha, Avena sativa, Candida utilis, Cladesporium fulvum, Claviceps purpurea, Cochiliobolus sativus, Coriolus versicolor, Corlinellus shiitake, Corticium vagum, Grifola umbellate, Hordeum vulgare, Pichia fermentans, Poria cocos, Sclerotium coffeicolum, Sclerotium delphnii, Sclerotium glucanium, Sclerotium rolsfi, Secale cereal, Shizophyllum commune, Streptococcus salvarius, Stereum sanguinolentum, Wingea robertsii, Lentines edodes, Sclerotium Acremonium diospyri, Monilinia fructigena, Aspergillus oryzae, Pestalotiopsis sp., Triticum sp., euglena cells, or a combination of any two or more thereof.

3. The wound dressing of claim 1 or claim 2, wherein the polysaccharide comprises oxidized regenerated cellulose (ORC), chitin, chitosan, an alginate, methyl cellulose, carboxymethyl cellulose, a glycosaminoglycan, chondroitin sulfate, heparin sulfate, or a combination of any two or more thereof.

4. The wound dressing of any one of claims 1-3, wherein the polysaccharide is oxidized regenerated cellulose (ORC) with a weight-average molecular weight of about 50,000 to about 1,000,000.

5. The wound dressing of any one of claims 1-4, wherein a weight ratio of the collagen to the polysaccharide is about 60:40 to about 40:60.

6. The wound dressing of any one of claims 1-5, wherein the collagen of the wound dressing is a mammalian collagen.

7. The wound dressing of claim 6, wherein the mammalian collagen is selected from the group consisting of a bovine collagen, a human collagen, a recombinantly derived collagen, and a combination of any two or more thereof.

8. The wound dressing of any one of claims 1-7, further comprising a silver compound.

9. The wound dressing of claim 8, wherein the wound dressing comprises about 0.1 wt. % to about 3 wt. % of the silver compound.

10. The wound dressing of claim 8 or claim 9, wherein the silver compound comprises one or more pharmaceutically acceptable silver salts.

11. The wound dressing of claim 10, wherein the one or more pharmaceutically acceptable silver salts comprises one or more of silver oxide, silver chromate, silver allantoinate, silver borate, silver glycerolate, silver nitrate, silver acetate, silver chloride, silver sulfate, silver lactate, silver bromide, silver iodide, silver carbonate, silver citrate, silver laurate, silver deoxycholate, silver salicylate, silver p-aminobenzoate, silver p-aminosalicylate, nanocrystalline silver, any pharmaceutically acceptable salt thereof, or a combination of any two or more thereof.

12. The wound dressing of any one of claims 1-11, wherein the wound dressing has a thickness of about 0.5 mm to about 5 mm.

13. The wound dressing of any one of claims 1-12, wherein the wound dressing further comprises one or more additional biomaterials, an antioxidant, a plasticizer, a growth factor, or a combination of any two or more thereof.

14. The wound dressing of claim 13, wherein the additional biomaterials are selected from the group consisting of gelatin, fibronectin, and a combination of any two or more thereof.

15. The wound dressing of claim 13 or claim 14, wherein the wound dressing comprises about 0.1 wt. % to about 10 wt. % of the additional biomaterials.

16. The wound dressing of any one of claims 13-15, wherein the antioxidant comprises one or more of anthocyanins, astaxanthin, bilirubin, canthaxanthin, capsaicin, citric acid, curcumin, coenzyme Q10, eugenol, flavanols, flavonolignans, flavanones, flavones, flavonols, iodide, isoflavone phytoestrogens, lutein, lycopene, manganese, melatonin, N-acetylcysteine, oxalic acid, phenolic acids, phytic acid, R-α-lipoic acid, stilbenoids, tocopherol, tocotrienol, vitamin A, vitamin C, vitamin E, xanthones, zeaxanthin, α-carotene, β-carotene, or a combination of any two or more thereof.

17. The wound dressing of any one of claims 13-16, where the wound dressing comprises about 0.1 wt. % to about 10 wt. % of the antioxidant.

18. The wound dressing of any one of claims 13-17, wherein the plasticizer comprises an acetylated monoglyceride, an alkyl citrate, methyl ricinoleate, glycerol, or a combination of any two or more thereof.

19. The wound dressing of claim 18, wherein the alkyl citrate is one or more of triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, or a combination of any two or more thereof.

20. The wound dressing of any one of claims 13-19, wherein the wound dressing comprises about 1 wt. % to about 10 wt. % of the plasticizer.

21. The wound dressing of any one of claims 13-20, wherein the wound dressing comprises about 1 wt. % to about 10 wt. % of the growth factor.

22. The wound dressing of any one of claims 13-21, wherein the growth factor comprises one or more of an epidermal growth factor (EGF), a hepatocyte growth factor (HGF), a fibroblast growth factor (FGF), a platelet-derived growth factor (PDGF), or a combination of any two or more thereof.

23. The wound dressing of claim 22, wherein the fibroblast growth factor comprises one or more of fibroblast growth factor 1 (FGF1), fibroblast growth factor 2 (FGF2), fibroblast growth factor 3 (FGF3), fibroblast growth factor 4 (FGF4), fibroblast growth factor 5 (FGF5), fibroblast growth factor 6 (FGF6), fibroblast growth factor 7/keratinocyte growth factor (FGF7/KGF), fibroblast growth factor 8 (FGF8), fibroblast growth factor 9 (FGF9), fibroblast growth factor 10/keratinocyte growth factor 2 (FGF10/KGF2), fibroblast growth factor 11 (FGF11), fibroblast growth factor 12 (FGF12), fibroblast growth factor 13 (FGF13), fibroblast growth factor 14 (FGF14), fibroblast growth factor 15 (FGF15), fibroblast growth factor 16 (FGF16), fibroblast growth factor 17 (FGF17), fibroblast growth factor 18 (FGF18), fibroblast growth factor 19 (FGF19), fibroblast growth factor 20 (FGF20), fibroblast growth factor 21 (FGF21), fibroblast growth factor 22 (FGF22), fibroblast growth factor 23 (FGF23), or a combination of any two or more thereof.

24. A method for treating a wound in a subject in need thereof, comprising administering to the wound a wound dressing of any one of claims 1-23.

25. The method of claim 24, wherein administering the wound dressing stimulates cytokine production.

26. The method of claim 24 or claim 25, wherein administering the wound dressing stimulates growth factors.

27. The method of any one of claims 24-26, wherein administering the wound dressing provides a reduction in the degradation of cytokines.

28. The method of any one of claims 24-27, wherein administering the wound dressing provides a reduction in the degradation of growth factors.

29. The method of any one of claims 24-28, wherein administering the wound dressing provides an inhibition of proteases.

30. The method of any one of claims 24-29, wherein the wound is an acute wound or a chronic wound.

31. The method of any one of claims 24-30, wherein the wound dressing is administered directly to the wound.

32. The method of any one of claims 24-31, wherein treating the wound comprises negative-pressure therapy.

33. The method of any one of claims 24-32, wherein the method further comprising administering negative pressure to the wound subsequent to administering the wound dressing.

34. A kit comprising the wound dressing of any one of claims 1-23, and instructions for use.

35. A kit comprising
a dressing and instructions for making the wound dressing of any one of claims 1-23; wherein dressing comprises
about 30 wt. % to about 95 wt. % of a collagen; and
about 30 wt. % to about 70 wt. % of a polysaccharide.
